(19) United States
(12) Patent Application Publication
(10) Pub. No.: US 2023/0135002 A1
(43) Pub. Date: May 4, 2023

(54) METHODS OF PROFILING TRANSLATION RATE

(71) Applicant: The Regents of the University of California, Oakland, CA (US)

(72) Inventors: Eugene Yeo, La Jolla, CA (US); Eric Van Nostrand, La Jolla, CA (US)

(21) Appl. No.: 17/915,167

(22) PCT Filed: Mar. 30, 2021
(86) PCT No.: PCT/US2021/024904

§ 371 Date: Sep. 28, 2022

Related U.S. Application Data
(60) Provisional application No. 63/002,833, filed on Mar. 31, 2020.

(51) Int. Cl. C12Q 1/6869; C12Q 1/6804

(52) U.S. Cl. CPC C12Q 1/6869; C12Q 1/6804; C12Q 1/6844

(57) ABSTRACT

Provided are methods of determining RNA translation rate in a biological sample, for example by crosslinking an RNA binding protein (RBP) to an RNA in a biological sample; identifying an RNA-RNA binding protein (RBP) complex within the biological sample, wherein the RNA-RBP complex comprises an RNA fragment bound by the RNA binding protein; and profiling the RNA fragment bound by the RNA binding protein, thereby determining a translation rate of the RNA in the biological sample.

### CROSS-REFERENCE TO RELATED APPLICATIONS

This application claims priority to U.S. Provisional Application Ser. No. 63/002,833, filed on Mar. 31, 2020. The disclosure of the prior application is considered part of the disclosure of this application, and is incorporated herein by reference in its entirety.

### FEDERALLY SPONSORED RESEARCH OR DEVELOPMENT

This invention was made with Government support under Grant No. HG004659 and HG009530 awarded by the National Institutes of Health. The Government has certain rights in the invention.

### BACKGROUND

Gene expression is often measured by quantification of protein level and/or RNA abundance. However, direct quantification of protein abundance in an unbiased and genome-wide manner remains technically challenging.

### SUMMARY

The present disclosure is based, at least in part, on quantifying the translation rate RNAs and individual genes.

Provided herein are methods of determining RNA translation rate in a biological sample, the method comprising: (a) crosslinking an RNA binding protein (RBP) to an RNA in a biological sample; (b) identifying an RNA-RNA binding protein (RBP) complex within the biological sample, wherein the RNA-RBP complex comprises an RNA fragment bound by the RNA binding protein; and (c) profiling the RNA fragment bound by the RNA binding protein, thereby determining a translation rate of the RNA in the biological sample.

In some embodiments, the crosslinking comprises UV crosslinking. In some embodiments, the method further comprises lysing the biological sample and fragmenting the RNA in the biological sample after step (a), thereby producing a lysate comprising a plurality of RNA fragments. In some embodiments, the fragmenting of the RNA comprises using a nuclease. In some embodiments, the nuclease comprises an RNase.

In some embodiments, identifying step (b) further comprises contacting the RNA-RBP complex with an RBP specific antibody, thereby allowing immunoprecipitation of the RNA-RBP complex with the RBP specific antibody bound to the RNA-RBP complex. In some embodiments, the immunoprecipitation of the RNA-RBP complex comprises using a bead to specifically bind the RBP specific antibody. In some embodiments, identifying step (b) further comprises dephosphorylating a 3′ and a 5′ end of the RNA fragment and ligating an RNA adapter to the 3′ end of the RNA fragment.

In some embodiments, profiling step (c) comprises isolating the RNA fragment of the immunoprecipitated RNA-RBP complex and producing a cDNA fragment by reverse transcription. In some embodiments, profiling step (c) further comprises ligating a DNA adapter to a 3′ end of the cDNA fragment; amplifying the RNA fragment or the cDNA fragment to generate one or more amplification products; and sequencing the one or more amplification products to identify the isolated RNA fragment and determine the translation rate of the RNA in the biological sample. In some embodiments, profiling step (c) further comprises depleting rRNA-containing amplification products before sequencing the one or more amplification products. In some embodiments, the sequencing comprises high-throughput sequencing. In some embodiments, the method further comprises removing a portion of the lysate prior to the immunoprecipitation and isolating the plurality of RNA fragments in the portion of the lysate.

Also provided herein are methods of analyzing RNA translation rate in a biological sample, the method comprising: (a) identifying an RNA-RNA binding protein (RBP) complex within a biological sample, wherein the RNA-RBP complex comprises an RNA fragment bound by an RNA binding protein; (b) isolating the RNA-RBP complex; and (c) profiling the RNA fragment bound by the RNA binding protein, thereby analyzing RNA translation rate in the biological sample.

In some embodiments, isolating step (b) comprises contacting the RNA-RBP complex with an RBP specific antibody, thereby allowing immunoprecipitation of the RNA-RBP complex with the RBP specific antibody bound to the RNA-RBP complex. In some embodiments, the immunoprecipitation of the RNA-RBP complex comprises using a bead to specifically bind the RBP specific antibody. In some embodiments, isolating step (b) further comprises dephosphorylating a 3′ and a 5′ end of the RNA fragment and ligating an RNA adapter to the 3′ end of the RNA fragment.

In some embodiments, profiling step (c) comprises isolating the RNA fragment of the immunoprecipitated RNA-RBP complex and producing a cDNA fragment by reverse transcription. In some embodiments, profiling step (c) further comprises ligating a DNA adapter to a 3′ end of the cDNA fragment; amplifying the RNA fragment or the cDNA fragment to generate one or more amplification products; and sequencing the one or more amplification products to identify the isolated RNA fragment and determine the translation rate of the RNA in the biological sample. In some embodiments, profiling step (c) further comprises depleting rRNA-containing amplification products before sequencing the one or more amplification products. In some embodiments, the sequencing comprises high-throughput sequencing.

In some embodiments, the RNA binding protein comprises a ribosomal protein. In some embodiments, the ribosomal protein is RPS3 or RPS2.

In some embodiments, the biological sample is a tissue sample. In some embodiments, the tissue sample comprises live cells from a cell culture. In some embodiments, the tissue sample is a fresh, frozen tissue sample. In some embodiments, the fresh, frozen tissue sample is cryoground into powder.

Provided herein are kits comprising: (a) a lysing agent, wherein the lysing agent lyses the biological sample and fragments RNA in a biological sample, thereby producing a lysate comprising a plurality of RNA fragments; (b) an RBP specific antibody, wherein the RBP specific antibody binds to an RNA-RBP complex, wherein the RNA-RBP complex comprises an RNA fragment bound by the RBP; and (c) a bead to specifically bind to the RBP specific antibody, wherein the bead allows immunoprecipitation of the RNA-RBP complex with the RBP specific antibody bound to the RNA-RBP complex.

In some embodiments, the lysing agent comprises a nuclease. In some embodiments, the nuclease comprises an RNase. In some embodiments, the kit further comprises: (d) a dephosphorylation agent, wherein the dephosphorylation agent dephosphorylates a 3′ and a 5′ end of the RNA fragment; (e) an RNA adapter, wherein the RNA adapter ligates to the 3′ end of the RNA fragment; and (f) a DNA adapter, wherein the DNA adapter ligates to a 3′ end of a cDNA fragment, wherein the cDNA fragment is produced by reverse transcription of the RNA fragment.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 shows an exemplary schematic of ribosomal protein enhanced crosslinking and immunoprecipitation (Ribo-eCLIP), wherein a biological sample (e.g., cells, tissues) is crosslinked and the sample is then lysed and RNA in the biological sample is fragmented with RNase, after which a ribosomal subunit (e.g., RPS3 or RPS2) is immunoprecipitated using an antibody which pulls down RPS3 and the crosslinked RNA. The RNA is dephosphorylated and an RNA adapter is ligated to the 3′ end. RNA is then isolated by Proteinase treatment followed by standard RNA column purification, and then reverse transcribed to cDNA, after which a second adapter is ligated to the 3′ end of the cDNA. Samples are then PCR amplified and subjected to high-throughput sequencing.

FIG. 2A shows a majority of reads for small ribosomal protein subunits (RPS) and large ribosomal protein subunits (RPL) eCLIP map to ribosomal RNA, but 1.1 million (out of 15.2 million total) map to exonic regions for RPS3.

FIG. 2B shows RPS3 peak density indicating broad distribution across mature mRNA metagene with enrichment at start and stop codons.

FIG. 3A shows an exemplary schematic of Ribo-eCLIP. As done in standard eCLIP, live cells or cryoground tissues are crosslinked with UV, and then fragmented with RNase I. Immunoprecipitation is then performed using anti-RPS3 antibody. Samples are either run on denaturing protein gels, transferred to nitrocellulose membrane, and the size range from 30 to 95 kDa is cut out and RNA is isolated, or RNA is directly isolated from beads. After reverse transcription, a 3′ DNA adapter is ligated, and PCR amplification is performed. Finally, fragments containing ribosomal RNA sequence are depleted to generate final library, which is re-amplified for sequencing.

FIG. 3B shows exemplary bars indicating fraction of reads mapping to indicated ribosomal RNAs (marked as rRNA_extra, RNA28S, RNA18S), exonic coding sequence or 3′UTR regions (marked as unique_CDS, unique_3utr), or other loci (marked as other).

FIG. 3C shows genes were separated into 10 bins based on translation rate estimate from polysome fractionation. For each bin, boxplot indicates 25^{th} to 75^{th} percentile for RPS3 eCLIP IP versus input ratio for the same set of genes.

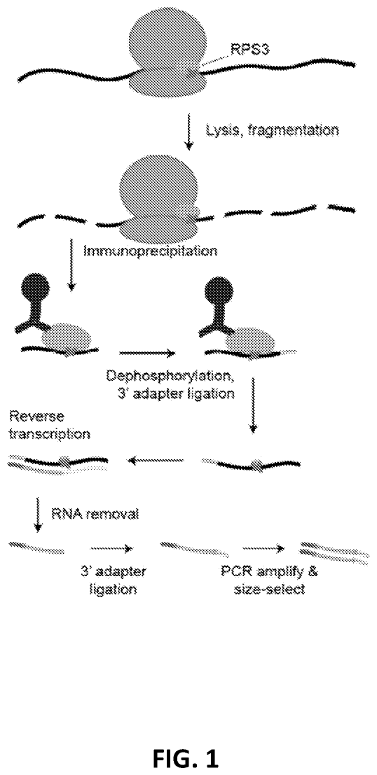

### DETAILED DESCRIPTION

This disclosure describes methods for profiling the translation rate of individual genes within a cell. Provided herein are methods of determining RNA translation rate in a biological sample, the method comprising: (a) crosslinking an RNA binding protein (RBP) to an RNA in a biological sample; (b)identifying an RNA-RNA binding protein (RBP) complex within the biological sample, wherein the RNA-RBP complex comprises an RNA fragment bound by the RNA binding protein; and (c) profiling the RNA fragment bound by the RNA binding protein, thereby determining a translation rate of the RNA in the biological sample.

In some embodiments, the translation rate of an RNA can be dynamic, with cis-regulation through RNA modifications and structures. In some embodiments, the translation rate of an RNA can be dynamic, with trans-regulation by RNA binding proteins which bind to RNA elements and can affect both initiation and elongation of translation by the ribosome. In some embodiments, the methods described herein determine the translation rate of only an actively translating RNA.

As used herein, “biological sample” can refer to a sample generally including cells and/or other biological material. A biological sample can be obtained from a mammalian organism. For example, a biological sample can be obtained from a human. A biological sample can be obtained from a non-human mammal (e.g., a dog, a cat, a monkey, a mouse, or a rat). A biological sample can be obtained from non-mammalian organisms (e.g., a plants, an insect, an arachnid, a nematode), a fungi, an amphibian, or a fish (e.g., zebrafish). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). Biological samples can be derived from a homogeneous culture or population of organisms or alternatively from a collection of several different organisms, for example, in a community or ecosystem.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

In some embodiments, the biological sample can be a tissue sample. In some embodiments, the tissue sample can include live cells from a cell culture. In some embodiments, the tissue sample can be a fresh, frozen tissue sample. In some embodiments, the fresh, frozen tissue sample is cryoground into powder. In some embodiments, the biological sample can be live cells on standard tissue culture dishes. In some embodiments, the biological sample can be flash, frozen tissues that have been cryoground into powder and placed on tissue culture dishes, pre-chilled on dry ice.

### RNA Binding Protein

RNA binding proteins (RBPs) are proteins that bind to the double or single stranded RNA in cells and have important roles in cellular processes (e.g., cellular transport, or localization). RBPs also play a role in post-transcriptional control of RNAs, such as RNA splicing, polyadenylation, mRNA stabilization, mRNA localization, and translation. In some embodiments, an RBP is a cytoplasmic protein. The term “RNA binding protein” can refer to a protein that interacts with RNA molecules (e.g., mRNA) from synthesis to decay to affect their metabolism, localization, stability, and translation. In some embodiments, an RBP is a nuclear protein. In some embodiments, RBPs can include, but are not limited to, splicing factors, RNA stability factors, histone stem-loop binding proteins, or ribosomes. For example, a eukaryotic ribosome can include a collection of RBPs that can interact directly with mRNA coding sequences.

In some embodiments, an RNA binding protein comprises a ribosomal protein, wherein the ribosomal protein binds to a ribosome and an mRNA during translation. In some embodiments, an RNA binding protein comprises a ribosomal protein, wherein the ribosomal protein binds to a ribosome or an mRNA during translation. In some embodiments, the RNA binding protein comprises at least one of: RBFOX1 (A2BP1), RBFOX2 (RBM9), RBFOX3 (NeuN), SLBP, RBM5, RBM6, PRBP1, ACO1, Adat1, PCBP1, PCBP2, PCBP3, PCBP4, RBM3, RBM4, RBM 5, RBM6, and APOBEC1. In some embodiments, the RNA binding protein can comprise a ribosomal protein. In some embodiments, the ribosomal protein can be RPS3 or RPS2.

RNA-binding proteins (RBPs) have roles in controlling the fate of RNAs including the modulation of pre-mRNA splicing, RNA modification, translation, stability and localization. RBPs are a group of proteins that interact with RNA using an array of strategies from well-defined RNA-binding domains to disordered regions that recognize RNA sequence and/or secondary structures.

As used herein, “RNA-RBP complex” can refer to a ribonucleoprotein complex comprising an RNA-binding protein (RBP) bound to a double or single stranded RNA in a cell. In some embodiments, the RNA-RBP complex can include an RNA fragment bound by an RNA binding protein. In some embodiments, the RBP is crosslinked to an RNA in a biological sample. In some embodiments, the crosslinking can include UV crosslinking. In some embodiments, the RBP is covalently linked to the RNA in a biological sample. In some embodiments, crosslinking can be performed by any method including, but not limited to, thermal crosslinking, chemical crosslinking, physical crosslinking, ionic crosslinking, photo-crosslinking, free-radical initiation crosslinking, an addition reaction, condensation reaction, water-soluble crosslinking reactions, irradiative crosslinking (e.g., x-ray, electron beam), or combinations thereof.

As used herein, “ribosomal protein” can refer to a protein that is present in a ribosome (e.g., a mammalian ribosome) or a protein that binds to a ribosome and an mRNA during translation (e.g., a translation initiation factor, a translation elongation factor, and a translation termination factor). The eukaryotic ribosome is composed of 79 ribosomal proteins, large ribosomal proteins (RPLs) and small subunit proteins (RPSs) that interweave with 4 highly structured RNAs (5S, 5.8S, 18S, and 28S rRNAs) to form the final translation-capable ribonucleoprotein. Thus, quantification of ribosome-associated RNA is highly similar to profiling of RNAs associated with other RNA binding proteins.

In some embodiments, the ribosomal protein binds to a ribosome or an mRNA during translation. The term “translation initiation factor” can refer to a protein that binds to a ribosome, a subunit of a ribosome, and/or an mRNA during the start of translation of an mRNA. The term “translation elongation factor” can refer to a protein that binds to a ribosome, a subunit of a ribosome, and/or mRNA during translation of an mRNA. The term “translation termination factor” can refer to a protein that binds to a ribosome, a subunit or a ribosome, and/or mRNA during cessation of translation and/or release of an mRNA from a ribosome or a subunit of a ribosome. In a ribosome, ribosomal proteins can participate in the translation process and binding of translation factors (e.g., translation initiation factor, translation elongation factor, translation termination factor). In some embodiments, the ribosomal protein is selected from the group consisting of: RPS2, RPS3, RPS3A, RPS4X, RPS4Y1, RPS4Y2, RPS5, RPS6, RPS7, RPS8, RPS9, RPS10, RPS11, RPS12, RPS13, RPS14, RPS15, RPS15A, RPS16, RPS17, RPS18, RPS19, RPS20, RPS21, RPS23, RPS24, RPS25, RPS26, RPS27, RPS28, RPS29, RPS30, RSSA, RACK1, RPL3, RPL4, RPL5, RPL6, RPL7A, RPL7, RPL8, RPL9, RPL10A, RPL10, RPL11, RPL12, RPL13A, RPL13, RPL14, RPL15, RPL17, RPL18A, RPL18, RPL19, RPL21, RPL22, RPL23A, RPL23, RPL24, RPL26, RPL27A, RPL27, RPL28, RPL29, RPL30, RPL31, RPL32, RPL34, RPL35A, RPL35, RPL36, RPL37A, RPL37, RPL38, RPL39, RPL40, RPL41, RPLA0, RPLA1, and RPLA2. In some embodiments, the ribosomal protein is a translation initiation factor. In some embodiments, the ribosomal protein is a translation elongation factor. In some embodiments, wherein the ribosomal protein is a translation termination factor.

### Ribo-eCLIP

Enhanced crosslinking and immunoprecipitation (eCLIP) is a method to profile RNAs bound by an RNA binding protein of interest. In some embodiments, eCLIP can be modified and used to profile RNAs bound by specific ribosomal subunit proteins (Ribo-eCLIP). As used herein, “Ribo-eCLIP” can refer to a method to profile ribosome-associated mRNAs. In some embodiments, a method of Ribo-eCLIP to determine RNA translation rate in a biological sample can include: (a) crosslinking an RNA binding protein (RBP) to an RNA in a biological sample; (b) identifying an RNA-RNA binding protein (RBP) complex within the biological sample, wherein the RNA-RBP complex comprises an RNA fragment bound by the RNA binding protein; and (c) profiling the RNA fragment bound by the RNA binding protein, thereby determining a translation rate of the RNA in the biological sample. In some embodiments, the method further comprises lysing the biological sample and fragmenting the RNA in the biological sample after step (a), thereby producing a lysate comprising a plurality of RNA fragments. In some embodiments, identifying step (b) further comprises contacting the RNA-RBP complex with an RBP specific antibody, thereby allowing immunoprecipitation of the RNA-RBP complex with the RBP specific antibody bound to the RNA-RBP complex. In some embodiments, the immunoprecipitation of the RNA-RBP complex comprises using a bead to specifically bind to the RBP specific antibody. In some embodiments, identifying step (b) further comprises dephosphorylating a 3′ and a 5′ end of the RNA fragment and ligating an RNA adapter to the 3′ end of the RNA fragment. In some embodiments, profiling step (c) comprises isolating the RNA fragment of the immunoprecipitated RNA-RBP complex and producing a cDNA fragment by reverse transcription. In some embodiments, profiling step (c) further comprises ligating a DNA adapter to a 3′ end of the cDNA fragment; amplifying the RNA fragment or the cDNA fragment to generate one or more amplification products; and sequencing the one or more amplification products to identify the isolated RNA fragment and determine the translation rate of the RNA in the biological sample. In some embodiments, profiling step (c) further comprises depleting rRNA-containing amplification products before sequencing the one or more amplification products. In some embodiments, the sequencing comprises high-throughput sequencing. In some embodiments, the method can further comprise removing a portion of the lysate prior to the immunoprecipitation and isolating the plurality of RNA fragments in the portion of the lysate.

Further, in some embodiments, a method of analyzing RNA translation rate in a biological sample can include: (a) identifying an RNA-RNA binding protein (RBP) complex within a biological sample, wherein the RNA-RBP complex comprises an RNA fragment bound by an RNA binding protein; (b) isolating the RNA-RBP complex; and (c) profiling the RNA fragment bound by the RNA binding protein, thereby analyzing

RNA translation rate in the biological sample. In some embodiments, the isolating step (b) comprises contacting the RNA-RBP complex with an RBP specific antibody, thereby allowing immunoprecipitation of the RNA-RBP complex with the RBP specific antibody bound to the RNA-RBP complex. In some embodiments, the immunoprecipitation of the RNA-RBP complex comprises using a bead to specifically bind to the RBP specific antibody. In some embodiments, the isolating step (b) further comprises dephosphorylating a 3′ and a 5′ end of the RNA fragment and ligating an RNA adapter to the 3′ end of the RNA fragment. In some embodiments, profiling step (c) comprises isolating the RNA fragment of the immunoprecipitated RNA-RBP complex and producing a cDNA fragment by reverse transcription. In some embodiments, profiling step (c) further comprises ligating a DNA adapter to a 3′ end of the cDNA fragment; amplifying the RNA fragment or the cDNA fragment to generate one or more amplification products; and sequencing the one or more amplification products to identify the isolated RNA fragment and determine the translation rate of the RNA in the biological sample. In some embodiments, profiling step (c) further comprises depleting rRNA-containing amplification products before sequencing the one or more amplification products. In some embodiments, the sequencing comprises high-throughput sequencing.

In some embodiments, enhanced crosslinking and immunoprecipitation (eCLIP) recovers protein-coding mRNAs, with a particular enrichment for coding sequence regions. In some embodiments, the normalized Ribo-eCLIP enrichment correlates with translation rate estimates from independent approaches. In some embodiments, Ribo-eCLIP enables mapping translation rate from a variety of cell lines and tissue models. In some embodiments, Ribo-eCLIP differs from the standard eCLIP method in that decreased RNA fragmentation is performed to enable improved depletion of ribosomal RNAs (rRNAs). In some embodiments, the fragmenting of the RNA can include using a nuclease. In some embodiments, the nuclease can include an RNase. In some embodiments, RNase fragmentation is performed with about 40U RNase I per 10 million cells. In some embodiments, RNase I can be decreased to about 38U RNase I per 10 million cells, 36U RNase I per 10 million cells, 34U RNase I per 10 million cells, 32U RNase I per 10 million cells, 30U RNase I per 10 million cells, 28U RNase I per 10 million cells, 26U RNase I per 10 million cells, 24U RNase I per 10 million cells, 22U

RNase I per 10 million cells, 20U RNase I per 10 million cells, 18U RNase I per 10 million cells, 16U RNase I per 10 million cells, 14U RNase I per 10 million cells, 12U RNase I per 10 million cells, 10U RNase I per 10 million cells, 8U RNase I per 10 million cells, 6U RNase I per 10 million cells, or 4U RNase I per 10 million cells. In some embodiments, RNase fragmentation can be performed with decreased RNase I of about 2U RNase I per 10 million cells.

In some embodiments, the depletion of fragments containing ribosomal RNA sequences is performed to enrich for ribosome-associated mRNAs. In some embodiments, immunoprecipitation is performed in Ribo-eCLIP using a validated antibody against a ribosomal subunit (e.g., RPS3 or RPS2) that can be crosslinked to an mRNA contained within the translating ribosome.

In some embodiments, isolating step (b) includes immunoprecipitation of the RNA-RBP complex. As used herein, “immunoprecipitation” is a technique of precipitating a protein antigen out of solution using an antibody that specifically binds to the protein antigen. In some embodiments, the solution containing the protein antigen is in the form of a crude lysate of an animal tissue. In some embodiments, the solution containing the protein antigen is in the form of a crude lysate of a plant tissue. In some embodiments, the solution can include a lysate from lysing a biological sample and fragmenting RNA in the biological sample, wherein the lysate includes a plurality of RNA fragments. In some embodiments, immunoprecipitation can be used to isolate and concentrate a specific protein from a biological sample containing many different proteins. In some embodiments, examples of immunoprecipitation can include, but are not limited to, protein complex immunoprecipitation (Co-IP), chromatin immunoprecipitation (ChIP), immunoprecipitation targeting ribonulceoproteins (RNP immunoprecipitation), or using a tagged protein (e.g., Green Fluorescent Protein (GFP), glutathione-S-tranferase (GST), or FLAG-tag) to bind with the antibody. In some embodiments, immunoprecipitation can be a direct capture method, wherein an antibody that specifically binds to a protein is attached to a substrate (e.g., superparamagnetic microbeads or agarose beads) such that the protein can be captured onto the substrate by the antibody. In some embodiments, immunoprecipitation can be an indirect capture method, wherein an antibody that specifically binds to a protein is contacted with the protein before attaching the antibody to a substrate (e.g., a bead coated with protein A/G).

In some embodiments, immunoprecipitation requires that the antibody be coupled to a solid substrate (e.g., immunoprecipitation beads) while preforming the procedure. In some embodiments, examples of the solid substrate can include, but are not limited to, agarose beads, superparamagnetic beads, Dynabeads magnetic beads, or streptavidin beads. In some embodiments, the substrate (e.g., immunoprecipitation beads) can be coated with Protein A, Protein G, Protein A/G, or Protein L.

In some embodiments, isolating step (b) further includes desphosphorylating a 3′ and a 5′ end of and RNA fragment and ligating an RNA adapter to the 3′ end of the RNA fragment. In some embodiments, dephosphorylation of an RNA fragment can include use of a phophatase to remove the phosphate of the 3′ and the 5′ end of the RNA fragment. In some embodiments, dephosphorylation is performed by using a phosphatase and a kinase. In some embodiments, the phosphatase can include an alkaline phosphatase (e.g., FastAP). In some embodiments, the kinase can include a T4 polynucleotide kinase (T4 PNK). In some embodiments, an RNA adapter in ligated to the 3′ end of the RNA fragment with an RNA ligase. In some embodiments, the RNA ligase is a T4 RNA ligase. In some embodiments, the RNA adapter can be barcoded. As used herein, a “barcode” is a label, or identifier, that conveys or is capable of conveying information (e.g., information about an RNA in a sample). In some embodiments, a barcode can be attached to an RNA adapter. In some embodiments, a particular barcode can be unique relative to other barcodes. In some embodiments, barcodes can have a variety of different formats. For example, barcodes can include non-random, semi-random, and/or random nucleic acid and/or amino acid sequences, and synthetic nucleic acid and/or amino acid sequences. In some embodiments, a barcode can be attached to an RNA fragment or to another moiety or structure in a reversible or irreversible manner. A barcode can be added to, for example, a fragment of a deoxyribonucleic acid (DNA) or ribonucleic acid (RNA) sample before or during sequencing of the sample.

In some embodiments, profiling step (c) comprises isolating the RNA fragment of the immunoprecipitated RNA-RBP complex and producing a cDNA fragment by reverse transcription. In some embodiments, the RNA fragment is isolated by using a protease to release the RNA fragment from the immunoprecipitation beads. In some embodiments, the protease is Proteinase K. In some embodiments, the isolated RNA fragment can have a total length of longer than 150 nucleotides (e.g., longer than 175 nucleotides, longer than 200 nucleotides, longer than 250 nucleotides, longer than 300 nucleotides, longer than 350 nucleotides, longer than 400 nucleotides, longer than 450 nucleotides, or longer than 500 nucleotides). In some embodiments, a cDNA fragment is produced by performing reverse transcription of the isolated RNA fragment. Reverse transcription can result in a cDNA transcript of the RNA fragment, which transcript includes each of the sequence segments of the nucleic acid molecule. In some embodiments, reverse transcription includes synthesizing cDNA from RNA (e.g., mRNA), using a reverse transcriptase.

As used herein, “profiling” can refer to the measurement of an activity (e.g., expression) of one or more genes, to create a global picture of cellular function. In some embodiments, profiling includes sequencing of a nucleic acid (e.g., DNA or RNA), wherein the gene expression profile includes information of active translation at a point in time. As used herein, the term “translation rate” can refer to the level of translation of an mRNA in a cell, wherein translation is the process in which ribosomes synthesize proteins after the process of transcription of DNA to RNA. In some embodiments, genetic differences and their subsequent expression as mRNAs impact the translation rate in an RNA-specific matter.

In some embodiments, profiling step (c) further includes ligating a DNA adapter to a 3′ end of the cDNA fragment; amplifying the RNA fragment or the cDNA fragment to generate one or more amplification products; and sequencing the one or more amplification products to identify the isolated RNA fragment and determine the translation rate of the RNA in the biological sample. In some embodiments, the RNA fragments or the cDNA fragments are amplified to yield quantities that are sufficient for analysis (e.g., via DNA sequencing). In some embodiments, amplification reaction is a polymerase chain reaction (PCR).

A wide variety of different sequencing methods can be used to analyze amplification products. Sequencing of polynucleotides can be performed by various commercial systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification. Other examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, MS-PET sequencing, and any combinations thereof.

In some embodiments, profiling step (c) further comprises depleting rRNA-containing amplification products before sequencing the one or more amplification products. In some embodiments, rRNA-containing amplification products can be depleted by using a ribosomal RNA depletion reagent. In some embodiments, depletion of rRNA-containing amplification products can be followed by additional amplification to enrich for mRNA-containing amplification products.

### Kits

Also provided herein are kits including: (a) a crosslinking agent, wherein the crosslinking agent crosslinks an RNA binding protein (RBP) to an RNA in a biological sample; (b) a lysing agent, wherein the lysing agent lyses the biological sample and fragments RNA in a biological sample, thereby producing a lysate comprising a plurality of RNA fragments; and (c) an RBP specific antibody, wherein the RBP specific antibody binds to an RNA-RBP complex, wherein the RNA-RBP complex comprises an RNA fragment bound by the RBP.

In some kits, the kit can further include a bead to specifically bind to the RBP specific antibody, wherein the bead allows immunoprecipitation of the RNA-RBP complex with the RBP specific antibody bound to the RNA-RBP complex. In some kits, the lysing agent comprises a nuclease. In some kits, the nuclease comprises an RNase.

In some kits, the kit can further include: (d) a dephosphorylation agent, wherein the dephosphorylation agent dephosphorylates a 3′ and a 5′ end of the RNA fragment; (e) an RNA adapter, wherein the RNA adapter ligates to the 3′ end of the RNA fragment; and (f) a DNA adapter, wherein the DNA adapter ligates to a 3′ end of a cDNA fragment, wherein the cDNA fragment is produced by reverse transcription of the RNA fragment.

In some kits, the RNA binding protein comprises a ribosomal protein. In some kits, the ribosomal protein is RPS3 or RPS2. In some kits, the biological sample is a tissue sample. In some kits, the tissue sample comprises live cells from a cell culture. In some kits, the tissue sample is a fresh, frozen tissue sample. In some kits, the fresh, frozen tissue sample is cryoground into powder.

### EXAMPLES

The disclosure is further described in the following examples, which do not limit the scope of the disclosure.

### Example 1—Sample Preparation

Samples (either live cells on standard tissue culture dishes, or flash frozen tissues cryoground into powder and placed on tissue culture dishes pre-chilled on dry ice) are UV crosslinked at 254 nm wavelength with 400 mJ/cm² to covalently link ribosomal proteins to bound RNAs. Cell samples are then scraped off of the plate, spun down (2000 g for 5 minutes), supernatant is removed, and cell pellets are flash-frozen and stored at −80° C. until use. Cryo-ground tissue is scraped off the plate (using pre-chilled sterile razor blades) into pre-chilled 1.5 mL tubes and stored at −80° C. until use.

### Example 2—Ribo-eCLIP

Cell pellets are lysed by adding 1 mL (per cell pellet of 10 million cells) of 4° C. eCLIP lysis buffer (50 mM Tris-HCl pH 7.4, 100 mM NaCl, 1% NP-40 (Igepal CA630), 0.1% SDS, 0.5% sodium deoxycholate) plus 10 μL Murine RNase Inhibitor (or equivalent), followed by incubation on ice for 5 minutes. Samples are then sonicated (e.g., Bioruptor on ‘low’ setting, 4° C., 5 min, 30sec on/30 sec off). To each sample is then added 10 μL containing 2U of RNase I (Ambion) diluted in 1×PBS, and 5 Turbo DNase, and samples are immediately placed at 37° C. for 5 minutes on a Thermomixer (1200 rpm shaking). After incubation, samples are centrifuged (15,000 g, 4° C., 3 min), and supernatant (containing cleared lysate) is moved to a new tube (pellet is discarded).

For each 10 million cells, 125 μL of Dynabeads (either sheep anti-rabbit or protein G) are washed twice in cold eCLIP lysis buffer and resuspeded in 100 μL eCLIP lysis buffer. 10 82 g of anti-RPS3 or RPS2 antibody is then added to each, and rotated at room temperature for 45 minutes. Following incubation, antibody-coupled beads are washed twice in cold eCLIP lysis buffer. Cleared lysate (from above) is then added to beads, and rotated overnight at 4° C.

Following overnight incubation, samples are washed twice with 500 μL of 4° C. eCLIP High Salt wash buffer (50 mM Tris-HCl pH 7.4, 1 M NaCl, 1 mM EDTA, 1% NP-40, 0.1% SDS, 0.5% sodium deoxycholate) and 500 μL of 4° C. eCLIP wash buffer (20 mM Tris-HCl pH 7.4, 10 mM MgCl2, 0.2% Tween-20, 5 mM NaCl), with buffer removed after each wash by magnetic separation of beads. Following washes, each sample is treated with FastAP (ThermoFisher) and T4 PNK (NEB) to dephosphorylate 3′ and 5′ ends of the RNA. Next, a barcoded RNA adapter was ligated to the 3′ end (T4 RNA Ligase, NEB) (at this step, multiple replicates of the same RBP, or potentially RBPs of similar size and bound RNA amount, can be uniquely barcoded and pooled after ligation to simplify downstream steps).

To increase signal and decrease background, samples can be run on standard protein gels and transferred to nitrocellulose membranes, and a region 75 kDa (˜150 nt of RNA) above the protein size can be isolated and Proteinase K (NEB) treated to isolate RNA. Alternatively, samples can be treated with Proteinase K (NEB) to release RNA from beads. In both cases, RNA is isolated by standard RNA column cleanup. After isolation, RNA is reverse transcribed (Superscript III, ThermoFisher), and treated with ExoSAP-IT (Affymetrix) to remove excess oligonucleotides. A second DNA adapter (containing a random-mer of 10 (N₁₀) random bases at the 5′ end) can be then ligated to the cDNA fragment 3′ end (T4 RNA Ligase, NEB). After cleanup (Dynabeads MyOne

Silane, ThermoFisher), an aliquot of each sample can be first subjected to qPCR (to identify the proper number of PCR cycles), and then the remainder was PCR amplified (Q5, NEB) and size selected via agarose gel electrophoresis.

At this stage, samples are then treated with ribosomal RNA depletion reagent (Jumpcode), followed by additional PCR to enrich for mRNA-only fragments.

### Example 3—Quantification of Ribosome-Associated RNAs

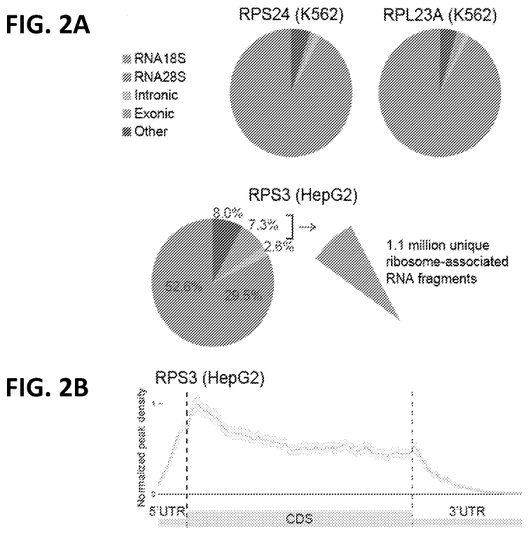

Focusing on RPS and RPL proteins, it was observed that a high fraction of reads mapped to ribosomal RNAs, with many showing greater than 90% of reads mapping either to the 18S or 28S rRNAs (FIG. 2A). Also, it was observed that more than 1 million uniquely mapped (non-PCR duplicate) reads at exons within protein-coding mRNAs from eCLIP of RPS3 (FIG. 2A). Moreover, meta-gene analysis indicated particular enrichment at the start and stop codons with a general decrease from the 5′ to 3′ direction, reminiscent of read density profiles observed in ribosome profiling data (FIG. 2B). These results are consistent with previous indications that RPS3 is a core component of the ribosome that lies proximal to (and thus can crosslink at high efficiency with) translating mRNA.

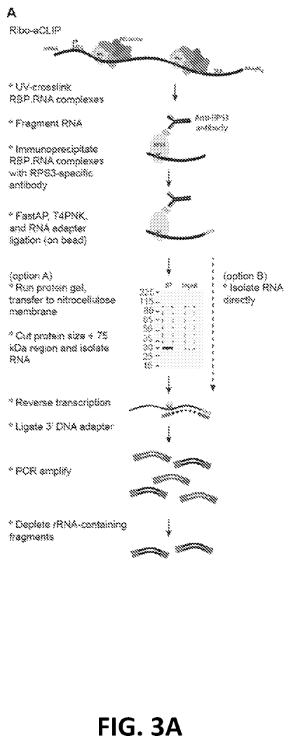

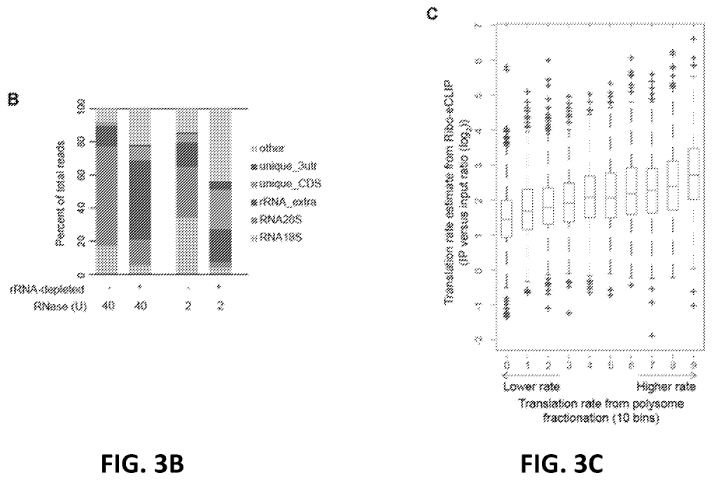

Additionally, modifications were performed to tailor the eCLIP protocol to profiling ribosome-associated mRNAs. This modified ‘Ribo-eCLIP’ approach differs from standard eCLIP in three distinct ways: (1) decreased RNase fragmentation is performed to enable improved depletion of ribosomal RNAs, (2) immunoprecipitation is performed using a validated antibody against a ribosomal subunit that crosslinks to mRNAs contained within the translating ribosome (currently RPS3 or RPS2), and (3) depletion of fragments containing ribosomal RNA sequences is performed to enrich for ribosome-associated mRNAs (FIG. 3A).

First, it was tested whether ribosomal RNA depletion could be incorporated into Ribo-eCLIP. Using standard eCLIP fragmentation conditions (40U RNase I per 10 million cells), it was observed that treatment with reagents to deplete ribosome-containing fragments showed limited depletion, likely due to the small average size of fragments in the library (FIG. 3B). To address this, the experiment was repeated with decreased RNase I (2U per 10 million cells), which increased average library fragment size. With this condition improved depletion was observed, with less than 5% of reads mapping to 18S or 28S ribosomal RNAs, and ˜25% of reads mapping to either coding or 3′ untranslated regions of mRNAs (FIG. 3B).

Next, it was tested whether Ribo-eCLIP accurately quantitates ribosome-associated RNAs and it was also tested whether per-gene enrichments in Ribo-eCLIP (quantitated as fold-enrichment in IP versus paired input) corresponded to independent measurement of ribosome-associated RNAs obtained by isolation of polysomes followed by RNA-seq. By binning all expressed RNAs into 10 bins based on the ratio of polysome-associated to monosome- or non-ribosome-associated expression, it was observed that Ribo-eCLIP enrichments indeed significantly correlated with polysome-associated RNA enrichments (FIG. 3C). Thus, Ribo-eCLIP recapitulates independent assessments of ribosome-associated RNAs.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining RNA translation rate in a biological sample, the method comprising:
(a) crosslinking an RNA binding protein (RBP) to an RNA in a biological ample;
(b) identifying an RNA-RNA binding protein (RBP) complex within the biological sample, wherein the RNA-RBP complex comprises an RNA fragment bound by the RNA binding protein; and
(c) profiling the RNA fragment bound by the RNA binding protein, thereby determining a translation rate of the RNA in the biological sample.

2. The method of claim 1, wherein the crosslinking comprises UV crosslinking.

3. The method of claim 1 or 2, wherein the method further comprises lysing the biological sample and fragmenting the RNA in the biological sample after step (a), thereby producing a lysate comprising a plurality of RNA fragments.

4. The method of claim 3, wherein the fragmenting of the RNA comprises using a nuclease.

5. The method of claim 4, wherein the nuclease comprises an RNase.

6. The method of any one of claims 1-5, wherein identifying step (b) further comprises contacting the RNA-RBP complex with an RBP specific antibody, thereby allowing immunoprecipitation of the RNA-RBP complex with the RBP specific antibody bound to the RNA-RBP complex.

7. The method of claim 6, wherein the immunoprecipitation of the RNA-RBP complex comprises using a bead to specifically bind the RBP specific antibody.

8. The method of any one of claims 1-7, wherein identifying step (b) further comprises dephosphorylating a 3′ and a 5′ end of the RNA fragment and ligating an RNA adapter to the 3′ end of the RNA fragment.

9. The method of any one of claims 6-8, wherein profiling step (c) comprises isolating the RNA fragment of the immunoprecipitated RNA-RBP complex and producing a cDNA fragment by reverse transcription.

10. The method of claim 9, wherein profiling step (c) further comprises ligating a DNA adapter to a 3′ end of the cDNA fragment; amplifying the RNA fragment or the cDNA fragment to generate one or more amplification products; and sequencing the one or more amplification products to identify the isolated RNA fragment and determine the translation rate of the RNA in the biological sample.

11. The method of claim 10, wherein profiling step (c) further comprises depleting rRNA-containing amplification products before sequencing the one or more amplification products.

12. The method of claim 10 or 11, wherein the sequencing comprises high-throughput sequencing.

13. The method of any one of claims 3-12, further comprising removing a portion of the lysate prior to the immunoprecipitation and isolating the plurality of RNA fragments in the portion of the lysate.

14. A method of analyzing RNA translation rate in a biological sample, the method comprising:
(a) identifying an RNA-RNA binding protein (RBP) complex within a biological sample, wherein the RNA-RBP complex comprises an RNA fragment bound by an RNA binding protein;
(b) isolating the RNA-RBP complex; and
(c) profiling the RNA fragment bound by the RNA binding protein, thereby analyzing RNA translation rate in the biological sample.

15. The method of claim 14, wherein isolating step (b) comprises contacting the RNA-RBP complex with an RBP specific antibody, thereby allowing immunoprecipitation of the RNA-RBP complex with the RBP specific antibody bound to the RNA-RBP complex.

16. The method of claim 15, wherein the immunoprecipitation of the RNA-RBP complex comprises using a bead to specifically bind the RBP specific antibody.

17. The method of any one of claims 14-16, wherein isolating step (b) further comprises dephosphorylating a 3′ and a 5′ end of the RNA fragment and ligating an RNA adapter to the 3′ end of the RNA fragment.

18. The method of any one of claims 15-17, wherein profiling step (c) comprises isolating the RNA fragment of the immunoprecipitated RNA-RBP complex and producing a cDNA fragment by reverse transcription.

19. The method of claim 18, wherein profiling step (c) further comprises ligating a DNA adapter to a 3′ end of the cDNA fragment; amplifying the RNA fragment or the cDNA fragment to generate one or more amplification products; and sequencing the one or more amplification products to identify the isolated RNA fragment and determine the translation rate of the RNA in the biological sample.

20. The method of claim 19, wherein profiling step (c) further comprises depleting rRNA-containing amplification products before sequencing the one or more amplification products.

21. The method of claim 19 or 20, wherein the sequencing comprises high-throughput sequencing.

22. The method of any one of claims 1-21, wherein the RNA binding protein comprises a ribosomal protein.

23. The method of claim 22, wherein the ribosomal protein is RPS3 or RPS2.

24. The method of any one of claims 1-23, wherein the biological sample is a tissue sample.

25. The method of claim 24, wherein the tissue sample comprises live cells from a cell culture.

26. The method of claim 24, wherein the tissue sample is a fresh, frozen tissue sample.

27. The method of claim 26, wherein the fresh, frozen tissue sample is cryoground into powder.

28. A kit comprising:
(a) a lysing agent, wherein the lysing agent lyses the biological sample and fragments RNA in a biological sample, thereby producing a lysate comprising a plurality of RNA fragments;
(b) an RBP specific antibody, wherein the RBP specific antibody binds to an RNA-RBP complex, wherein the RNA-RBP complex comprises an RNA fragment bound by the RBP; and
(c) a bead to specifically bind to the RBP specific antibody, wherein the bead allows immunoprecipitation of the RNA-RBP complex with the RBP specific antibody bound to the RNA-RBP complex.

29. The kit of claim 28, wherein the lysing agent comprises a nuclease.

30. The kit of claim 29, wherein the nuclease comprises an RNase.

31. The kit of any one of claims 28-30, further comprising:
(d) a dephosphorylation agent, wherein the dephosphorylation agent dephosphorylates a 3′ and a 5′ end of the RNA fragment;
(e) an RNA adapter, wherein the RNA adapter ligates to the 3′ end of the RNA fragment; and
(f) a DNA adapter, wherein the DNA adapter ligates to a 3′ end of a cDNA fragment, wherein the cDNA fragment is produced by reverse transcription of the RNA fragment.
